(19) United States
(12) Patent Application Publication
(10) Pub. No.: US 2025/0213549 A1
(43) Pub. Date: Jul. 3, 2025

(54) COMPOSITION COMPRISING NICOTINAMIDE, NICOTINAMIDE PRECURSORS, NICOTINAMIDE METABOLITES OR COMBINATIONS THEREOF FOR PREVENTING OR REDUCING ONE OR MORE POST-ACUTE SYMPTOMS OF INFECTIOUS DISEASES

(71) Applicant: CONARIS Research Institute AG, Kiel (DE)

(72) Inventors: Georg Wätzig, Kiel (DE); Stefan Schreiber, Kiel (DE)

(73) Assignee: CONARIS Research Institute AG, Kiel (DE)

(21) Appl. No.: 18/851,342

(22) PCT Filed: Mar. 29, 2023
(86) PCT No.: PCT/EP2023/058188

§ 371 Date: Sep. 26, 2024

(30) Foreign Application Priority Data
Mar. 30, 2022 (EP) 22165558.2

(51) Int. Cl. A61K 31/455; A61K 9/00; A61P 31/14

(52) U.S. Cl. CPC A61K 31/455; A61K 9/0014; A61K 9/0053; A61P 31/14

(57) ABSTRACT

A composition comprising an active substance selected from nicotinamide; nicotinic acid; nicotinic acid esters; tryptophan; a tryptophan dipeptide; nicotinamide adenine dinucleotide (NAD); nicotinamide adenine dinucleotide phosphate (NADP); an intermediate in the biosynthesis of NAD or NADP selected from the group consisting of N-formylkynurenine, L-kynurenine, 3-hydroxy-L-kynurenine, 3-hydroxyanthranilate, 2-amino-3-carboxymuconate semialdehyde, quinolinate, nicotinic acid mononucleotide (beta-nicotinate D-ribonucleotide), and nicotinic acid adenine dinucleotide; nicotinamide riboside; nicotinamide mononucleotide; 1-methylnicotinamide (N-methylnicotinamide); or a combination thereof, for use for preventing and/or reducing one or more post-acute symptoms of an infection, wherein the composition is formulated to partly or completely release the active substance(s), preferably nicotinamide, for topical supplementation or efficacy in the lower small intestine and/or the colon, wherein preferably the pathogen is SARS-CoV-2 and/or the post-acute symptoms are PCS

### FIELD OF THE INVENTION

The present invention relates to a composition comprising nicotinamide (niacinamide) or suitable precursors or metabolites thereof or combinations thereof for use for preventing or reducing post-acute symptoms of an infection—preferably long-term symptoms of the coronavirus disease 2019 (COVID-19) summarised under the term post-COVID syndrome (PCS).

### BACKGROUND

The disease course following infection with severe acute respiratory syndrome coronavirus type 2 (SARS-CoV-2) is highly variable, ranging from symptom-free infection to the development of severe acute respiratory syndrome with hospitalisation, eventual need for assisted ventilation and death. Reduced organ functions after recovery from acute COVID-19 may persist for weeks to months after the acute phase of the infection in patients following both mild and severe disease courses. There are different terms for this syndrome, such as PCS, post-acute COVID syndrome (PACS), post-acute sequelae of SARS-CoV-2 infection (PASC) or “long COVID”. In the present application, the term PCS is used. Compared to common respiratory viral infections like influenza with similar symptoms (e.g. cough, fever or fatigue), Persistent Somatic Symptoms are much more common and severe in COVID-19 patients, particularly after hospitalization (Marshall 2020, Nature 585:339; Groff et al. 2021, JAMA Netw. Open 4:e2128568; Jiang et al. 2021, JACC Basic Transl. Sci. 6:796). Nevertheless, post-acute symptoms of infectious diseases such as the post-infective fatigue syndrome have been described for more than 100 years, e.g. after the Spanish flu, and for a wide variety of viral, bacterial or protozoal pathogens, including—as non-limiting examples—Epstein-Barr virus, Dengue virus, Chikungunya virus, Ebola virus, West Nile virus, Coxiella burnetii, Cryptosporidium hominis and Giardia lamblia (Murray et al. 2014, PloS One 9:e10295; Rehn et al. 2015, BMC Public Health 15:529; Hung et al. 2019, Trends Parasitol. 35:673; Sandier et al. 2021, Open Forum Infect. Dis. 8:ofab440). Persistent Somatic Symptoms represent an umbrella term to describe subjectively distressing somatic complaints, irrespective of their aetiology, that are present on most days for at least several months after having recovered from an illness. Persistent Somatic Symptoms can be operationalised by repeated measures of patients' subjective somatic symptom severity and therefore include fatigue and other measures of suffering.

In COVID-19, which is preferably the infectious disease in the present application, the persistence of symptoms or tissue damage beyond the acute phase of the disease is the rule rather than the exception: for example, 88% of participants in a study with COVID-19 patients still had visual lung damage 6 weeks after their discharge from hospital, and in 56% the damage persisted at 12 weeks (Marshall 2020, Nature 585:339). A large meta-analysis showed that “more than half of COVID-19 survivors experienced PASC 6 months after recovery” (Groff et al. 2021, JAMA Netw. Open 4:e2128568). Key symptoms of PCS are fatigue (44% of patients), sleep disorder (33%), dyspnoea (40%), cough (22%) as well as anxiety (34%), depression (32%) and cognitive impairments (15%), and decreased quality of life was reported by 57% of PCS patients (Jennings et al. 2021, J. Clin. Med. 10:8913). Other meta-analyses found that the most frequent persisting symptoms of COVID-19 were fatigue, dyspnoea, exercise intolerance, impaired sense of smell and taste, cognitive impairments, headache, and general pain (Bahmer et al. 2022, eClinicalMedicine 51:101549). A recent large trial from Germany reported that “neurological ailments (61.5%), fatigue (57.1%), and sleep disturbance (57.0%) were the most frequent persisting symptoms at 6-12 months after infection” and developed a PCS score (Bahmer et al. 2022, eClinicalMedicine 51:101549). In this PCS score, 12 long-term symptom complexes (fatigue, cough/wheezing, neurological ailments, joint/muscle pain, ear-nose-throat ailments, gastrointestinal ailments, sleeping disturbance, exercise intolerance, infection signs, chemosensory deficits, chest pain and dermatological ailments) are combined with different weights.

For related coronaviruses like SARS-CoV-1, symptom persistence has also been described and has been shown to last for years (Marshall 2020, Nature 585:339). In any case, the persistence of symptoms is not restricted to hospitalized patients with severe COVID-19 as described above, but occurs in a similar fashion in patients with mild disease. For example, persistent fatigue following COVID-19 was independent from disease severity in a cohort of 128 patients from Ireland, of which 52.3% reported persistent fatigue at a median of 10 weeks after initial COVID-19 symptoms (Townsend et al. 2020, PloS ONE 15:e0240784). Moreover, not all symptoms of PCS or related conditions after other infections are dominant or even significantly prevalent in the acute phase (e.g., cognitive impairments), suggesting that the long-term syndromes resulting from such infections may be separate disease entities and are therefore not necessarily treatable with the same interventions that are efficacious in the acute phase of the infection.

Therefore, there is a large unmet need for active substances and compositions with no or minimal side effects that can prevent or reduce post-acute symptoms of infectious diseases (especially symptoms of PCS), thereby preventing or reducing especially the loss of quality of life which is very often reflected by impaired sense of smell and/or taste.

The term vitamin B3 comprises nicotinic acid and nicotinamide. In addition to being a precursor of the pivotal and ubiquitous coenzymes nicotinamide adenine dinucleotide (NAD) and its phosphorylated derivative nicotinamide adenine dinucleotide phosphate (NADP), nicotinamide is also involved in energy homoeostasis signalling pathways in intestinal epithelial cells and in maintaining the secretion of antimicrobial peptides from these cells (Hashimoto et al. 2012, Nature 487:477). With regard to maintaning the health and functionality of intestinal epithelial cells and the gut microbiota, nicotinamide has an efficacy similar to that of its precursor, the essential amino acid tryptophan (Hashimoto et al. 2012, Nature 487:477). Accordingly, sufficient amounts of tryptophan or nicotinamide are not only particularly important in fast replicating cells like epithelial cells to fuel energy metabolism, but supplementation of nicotinamide also protects from dysregulation of the intestinal microbiota and intestinal inflammation, particularly when the nicotinamide is topically delivered by appropriate formulations or compositions to the lower small intestine and large intestine where the microbiota are located (Hashimoto et al. 2012, Nature 487:477; Waetzig & Seegert 2013, PCT/EP2013/062363; Bettenworth et al. 2014, Mol. Nutr. Food Res. 58:1474; Wätzig & Seegert 2015, PCT/EP2014/077637; Wätzig & Seegert 2015, PCT/EP2014/077646). Moreover, supplementation of NAD precursors like nicotinamide has been suggested to be beneficial in fighting coronavirus infections (Heer et al. 2020, J. Biol. Chem. 295:17986). However, using nicotinamide is not the one and obvious choice, as nicotinamide riboside has been suggested to be more efficacious in raising NAD levels (Bogan-Brown et al. 2021, J. Diet. Suppl., DOI: 10.1080/19390211.2021.1881686). Nicotinamide is authorised for use in food [Regulation (EC) No 1925/2006, amended by Commission Regulation (EC) No 1170/2009], in food supplements (Directive 2002/46/EC) as well as in infant and follow-on formula, baby food and food for particular nutritional uses (Regulation (EU) No 609/2013). Nicotinamide is mainly marketed in the form of dietary supplements, although there are also nicotinamide prescription drugs for treating vitamin B3 deficiency. Nicotinamide has an excellent safety profile, resulting in a high Tolerable Upper Intake Level (UL) or lifelong Acceptable Daily Intake (ADI) of 12.5 mg/kg/d or 900 mg/d as defined by the European Food Safety Authority (EFSA 2002, SCF/CS/NUT/UPPLEV/39; EFSA 2014, EFSA J. 12:3759).

There is evidence for various virus types that nicotinamide can reduce viral replication and support the body's acute defence mechanisms, e.g., in the case of vaccinia virus (Child et al. 1988, Virus Res. 9:119), human immunodeficiency virus (Murray 2003, Clin. Infect. Dis. 36:453), enteroviruses (Moell et al. 2009, J. Med. Virol. 81:1082) or hepatitis B virus (Li et al. 2016, Arch. Virol. 161:621). Due to its putatively favourable risk/benefit ratio, sufficient supplementation of nicotinamide in COVID-19 patients has been recommended because of its antiviral activity and protective effects against lung injury and bacterial lung infections (Shi et al. 2020, Cell Death Differ. 27:1451; Gharote 2020, Ind. J. Med. Sci. 72:25; Zhang et al. 2020, J. Med. Virol. 92:479; Mehmel et al. 2020, Nutrients 12:1616; Shakoor et al. 2021, Maturitas 144:108; Brenner 2022, Nat. Metab. 4:2). Replenishment of NAD levels by NAM supplementation is considered to restore antiviral innate immune functions and rebalance the maladaptive, hyperinflammatory immune response to SARS-CoV-2 infection (Mehmel et al. 2020, Nutrients 12:1616).

The intestinal uptake of tryptophan, the precursor of nicotinamide, depends on the presence of angiotensin converting enzyme-2 (ACE2) on the intestinal epithelium (Hashimoto et al. 2012, Nature 487:477). ACE2 is required for SARS-CoV-2 to enter human body cells and is expressed at much higher levels in intestinal than in respiratory epithelia (Mitsuyama et al. 2020, J. Clin. Med. 9:3630). SARS-CoV-2 infection inevitably reduces the cell surface expression of ACE2, which leads to malabsorption of tryptophan, gut dysbiosis and intestinal inflammatory symptoms (Hashimoto et al. 2012, Nature 487:477; Mitsuyama et al. 2020, J. Clin. Med. 9:3630; Yeoh et al. 2021, Gut 70:698; Vanella et al. 2021, BMC Open Gastroenterol. 8:e000578; Sarkar et al. 2021, Trends Mol. Med. 27:1115). This could be compensated by administration of nicotinamide, the uptake of which does not depend on ACE2 and which is equally effective in maintaining intestinal health and a healthy gut microbiota (Hashimoto et al. 2012, Nature 487:477).

The rationale for elevating NAD levels in acute COVID-19 has recently been summarised (Brenner2022, Nat. Metab. 4:2) and the efficacy of nicotinamide in reducing acute symptoms of COVID-19 has been demonstrated (Watzig & Schreiber 2021, PCT/EP2021/083138). However, there are no published data on the long-term effects of nicotinamide or related substances on post-acute symptoms of infectious diseases (especially PCS). It is important to keep in mind that these symptoms partly differ from those in the acute phase of the respective disease (see above). Therefore, a person skilled in the art would not necessarily expect that interventions for the acute infection also prevent or ameliorate post-acute symptoms. On the other hand, a person skilled in the art would expect that an intervention that is effective against one or more symptoms in the acute phase of the disease would also be effective against the same symptom(s) in the long term, and that an intervention that is ineffective in the acute phase would also be ineffective in the long term.

Taken together, the state of the art suggests that nicotinamide or related chemicals as active substances in nutritional or pharmaceutical formulations can ameliorate the symptoms of COVID-19 during the first weeks after infection. However, as the severity of the acute disease course does not necessarily predict affliction by post-acute sequelae like PCS and as the acute and chronic conditions occur chronologically far apart and with partially different symptoms (see above), it was not to be expected by a person skilled in the art that dampening symptoms by supplementation of nicotinamide during the acute phase of an infectious disease (especially COVID-19) would have long-term beneficial effects on post-acute symptoms resulting from the infection (especially PCS). Moreover, it could not be expected that a substance like e.g. nicotinamide or related active substances would be ineffective against certain symptoms (e.g. impaired sense of smell and/or taste) in the acute phase of the disease, but become significantly effective against the same symptom(s) in the long term.

Therefore, it was an object of the present invention to provide means for reducing the risk of post-acute symptoms of infectious diseases (especially PCS). In addition to that, it was an object of the present invention to provide means for a pre- or post-exposure prophylaxis to prevent the onset of post-acute symptoms of infectious diseases (especially PCS) in patients that were at risk for an infection or tested positive for infection with a pathogen (especially SARS-CoV-2).

According to the invention, this object is solved by a composition comprising an active substance selected from nicotinamide; nicotinic acid; nicotinic acid esters; tryptophan; a tryptophan dipeptide; nicotinamide adenine dinucleotide (NAD); nicotinamide adenine dinucleotide phosphate (NADP); an intermediate in the biosynthesis of NAD or NADP selected from the group consisting of N-formylkynurenine, L-kynurenine, 3-hydroxy-L-kynurenine, 3-hydroxyanthranilate, 2-amino-3-carboxymuconate semialdehyde, quinolinate, nicotinic acid mononucleotide (beta-nicotinate 0-ribonucleotide), and nicotinic acid adenine dinucleotide; nicotinamide riboside; nicotinamide mononucleotide; 1-methylnicotinamide (N-methylnicotinamide); or a combination thereof, for use for preventing or reducing one or more post-acute symptoms of the infection (especially PCS), wherein the composition is formulated to partly or completely release the active substance, preferably nicotinamide, for topical supplementation or efficacy in the lower small intestine and/or the colon, wherein preferably the pathogen is SARS-CoV-2 and/or the post-acute symptoms are PCS.

Preferably, the composition according to the invention is formulated for oral administration.

The human body is a metaorganism incorporating human cells and a multitude of microbial species, particularly in the intestinal microbiota. The sum of all metabolic pathways available from all parts of this metaorganism has to be viewed as a whole and can be used by the human body either directly or indirectly. Tryptophan and nicotinic acid cannot be synthesised de novo by human cells, but can be taken up from diverse sources from the gut. The active substances listed above can be synthesised as intermediates in synthesis pathways leading from these precursors to NAD or NADP. Therefore, these substances are considered functionally equivalent to the particularly safe and well-characterised NAD(P) precursor nicotinamide. For example, in human nutrition science, the term “niacin equivalent” already indicates the interchangeability of the precursor tryptophan and its products nicotinic acid and nicotinamide (which are summarised as niacin or vitamin B3): approximately 60 mg of tryptophan yields 1 mg of niacin defined as 1 mg niacin equivalent (EFSA Scientific Opinion on dietary reference values for niacin; EFSA Journal 2014; 12:3759). For reasons of conciseness, the description and exemplification of the present invention focuses on nicotinamide without objective restriction to this compound. However, nicotinamide is the most preferred active substance.

Preferably, at least one post-acute symptom is selected from the group consisting of reduced physical performance (e.g., inability to perform normal activities, performance drop or reduced physical capacity), fatigue, exercise intolerance (e.g., shortness of breath, dyspnea or reduced exercise capacity), chemosensory deficits (impaired sense of smell or taste), joint pain, muscle pain, chest pain, ear-nose-throat ailments (e.g., hoarseness, sore throat or running nose) cough and/or wheezing, gastrointestinal ailments (e.g., abdominal pain, diarrhea, nausea or vomiting), neurological ailments (e.g., confusion, vertigo, headache, motor deficits, sensory deficits, numbness, dizziness, tremor, deficits of concentration, cognition or speech), psychological ailments (e.g., anxiety, depression, stress or reduced resilience), dermatological ailments (e.g., skin rash, itchiness or hair loss), infection signs (e.g., chills, fever, general sickness or flu-like symptoms), or sleep disturbance (e.g., insomnia, unrestful sleep), wherein the particularly preferred selected symptoms are impaired sense of smell and/or taste.

In the sense of this specification, symptoms may be self-assessed by patients (e.g., by interviews and/or questionnaires and/or mobile apps, particularly regarding multidimensional parameters of general well-being like the ability to perform normal activities) and/or by objective examinations and tests (e.g., by physical examinations to measure, e.g., shortness of breath, by electronic device monitoring of activities and/or physical parameters and/or, e.g., by smell tests to detect and/or quantify an impaired sense of smell). In case of doubt, the assessment of the respective symptoms is made via self-assessment.

The person skilled in art understands that in the sense of the present invention, a “resolution of symptoms” only can occur if such symptoms were present. As used herein, “resolution of symptoms” means that symptoms that where present were completely eliminated at least according to the impression of the patient.

According to the invention, the problem stated above is preferably solved by the use of nicotinamide in a supplementation or treatment regimen or a composition comprising nicotinamide, as defined in the claims and/or described in more detail herein. The use of suitable precursors or metabolites of nicotinamide, alone or in combination, together with or instead of nicotinamide, is also in the scope of the present invention. For reasons of conciseness, this is not repeated in all instances, but nicotinamide is used as a preferred example. In preferred embodiments, the composition comprising nicotinamide is formulated to partly or completely release the nicotinamide in the lower small intestine and/or the colon to beneficially and topically influence the intestinal mucosa and the intestinal microbiota as described in the following patent families: Waetzig & Seegert 2013, PCT/EP2013/062363; Wätzig & Seegert 2015, PCT/EP2014/077637; Wätzig & Seegert 2015, PCT/EP2014/077646; Schwarz et al. 2017, PCT/EP2017/058733. For example, nicotinamide is formulated to be released selectively, e.g., for at least partially topical efficacy, in the lower small intestine and/or colon, where the intestinal microbiota are located. Accordingly, compositions are provided which preferably contain nicotinamide which prevent or reduce one or more symptoms of PCS.

### BRIEF DESCRIPTION OF THE DRAWINGS

FIG. 1 shows the frequencies of impaired sense of smell and taste at the 6-month follow-up in patients of the risk factor population of the COVit-2 trial (percentage and 95% confidence interval).

FIG. 2 shows box plots of the PCS score in patients of the risk factor population of the COVit-2 trial with more than 5 predictors for PCS.

### DETAILED DESCRIPTION

In the futility analysis population (n=402) of the COVit-2 trial (Example 2; Wätzig & Schreiber 2021, PCT/EP2021/083138), the use of a combination of immediate-release and controlled-ileocolonic-release nicotinamide surprisingly led to significant improvements in the ability to perform normal activities, in physical performance and in reduction of fatigue in the first weeks after infection in patients with at least one characteristic or underlying medical condition associated with an increased risk of developing severe illness from COVID-19 as well as in patients with key symptoms of COVID-19. At 6 months after infection, patients were contacted for follow-up interviews to determine their affliction by PCS using a large battery of questionnaires and tests including antibody level measurements (Example 2). For the first 50 patients of each trial arm (nicotinamide vs. placebo), data quality control tests were performed and surprisingly revealed that nicotinamide supplementation—which ameliorated the acute disease course of COVID-19—also reduced symptoms of PCS even if the same symptoms were not reduced in the acute disease course. Especially, nicotinamide supplementation led to significantly reduced frequencies of impaired sense of smell or taste after six months, even though nicotinamide was clearly ineffective at influencing these symptoms in the acute phase of the disease.

Therefore, the core of the present invention is the use of the composition according to the invention preferably comprising nicotinamide in a supplementation or treatment regimen to prevent or reduce one or more post-acute symptoms of an infection (especially PCS), preferably in form of a composition fomulated for oral administration for this use, as defined in the claims and/or described in more detail herein. In particular, improvements in patients with at least one characteristic or underlying medical condition associated with an increased risk of developing severe illness from the infection (especially COVID-19) as well as in patients with at least one characteristic symptom of the infection are preferred effects of the invention. The use of the composition according to the invention preferably comprising nicotinamide as a pre- or post-exposure prophylaxis in case of an infection risk or after positive testing for infection with the pathogen (especially SARS-CoV-2) and before the onset of disease symptoms is also preferred within the scope of the present invention.

In addition to the preferred active substance nicotinamide, suitable precursors or metabolites of nicotinamide can be used in the invention as active substances. For example, compounds that convert into nicotinamide (e.g., by hydrolysis or metabolism) in the human or animal body are suitable, such as nicotinic acid or nicotinic acid esters. In addition, intermediates in the biosynthesis of nicotinamide adenine dinucleotide (NAD) or NAD phosphate (NADP) starting from tryptophan, such as N-formylkynurenine, L-kynurenine, 3-hydroxy-L-kynurenine, 3-hydroxyanthranilate, 2-amino-3-carboxymuconate semialdehyde, quinolinate, nicotinic acid mononucleotide (beta-nicotinate D-ribonucleotide), and nicotinic acid adenine dinucleotide, can be used. Further examples include NAD, NADP, nicotinamide riboside, nicotinamide mononucleotide or the nicotinamide metabolite 1-methylnicotinamide (N-methylnicotinamide). Dipeptidic tryptophan as an equivalent for nicotinamide in case of compromised ACE2 cell surface expression in the intestine (Hashimoto et al. 2012, Nature 487:477) is also in the scope of the present invention. The use of these suitable precursors or metabolites of nicotinamide, alone or in combination, together with or instead of nicotinamide, is also in the scope of the present invention. For reasons of conciseness, this is not repeated in all instances, but nicotinamide is used as a preferred example.

In general, the uses disclosed in this invention may be medical uses or non-medical uses. Medical use in the sense of the present application preferably means that the composition for use according to the invention is a medicament, authorised by the respective competent regulatory authority of the respective country where the use takes place, and wherein all other uses are non-medical uses.

It is preferred that the composition according to the invention is formulated for oral administration to partly or completely release nicotinamide for topical supplementation or efficacy in the lower small intestine and/or the colon to beneficially and topically influence the intestinal mucosa and the intestinal microbiota as described in the following patent families: Waetzig & Seegert 2013, PCT/EP2013/062363; Wätzig & Seegert 2015, PCT/EP2014/077637; Wätzig & Seegert 2015, PCT/EP2014/077646; Schwarz et al. 2017, PCT/EP2017/058733. Preferably, the composition according to the invention is formulated for at least partially selective release, more preferably for at least partially delayed release of nicotinamide for topical supplementation or efficacy, in the lower small intestine and/or colon, where the intestinal microbiota are located. Preferably, the composition according to the invention is formulated to start releasing nicotinamide at least partially in the second half of the jejunum. Alternatively preferred, the composition according to the invention is formulated to at least partially start releasing in the terminal ileum and/or colon. In a further preferred embodiment, the release of nicotinamide in both delayed and non-delayed dosage forms is prolonged by an extended-release and/or controlled-release formulation to achieve higher trough levels and a more constant systemic exposure.

In the present invention, the terms “formulation” or “composition” or “supplementation” or “treatment”, and in particular the term “composition”, have a broad meaning of a pharmaceutically and/or nutritionally and/or physiologically acceptable formulation, composition and/or mode of administration of nicotinamide, which includes, but is not limited to, medicaments (pharmaceutical formulations), medical products, nutraceuticals, foods for special medical purposes, dietary supplements, food ingredients and/or foods. The nature of the composition may vary, e.g., depending on the ingredients and excipients, the dose of nicotinamide, the formulation type and other factors. Preferred are dietary supplements, food for special medical purposes, nutraceuticals and medicaments.

The composition according to the invention preferably is formulated for at least partially delayed release of the active substance, preferably of nicotinamide, for topical supplementation or efficacy in the lower small intestine and/or the colon.

The composition according to the invention preferably comprises one or more active substance formulations, preferably nicotinamide formulations, for immediate release and/or extended release and/or sustained release delivering the active substance, preferably nicotinamide, mainly systemically to the circulation together with one or more active substance formulations, preferably nicotinamide formulations, for delayed release and/or delayed-controlled release delivering the active substance(s), preferably nicotinamide, mainly topically to the lower small intestine and/or colon. For definitions of delayed and delayed-controlled release, see below.

The composition according to the invention preferably contains a combination of two formulation variants of the active substance(s), preferably of nicotinamide, in a specific ratio by weight in the range of from 1:1 to 1:1000, preferably from 1:1 to 1:100, more preferably from 1:1 to 1:10. Alternative preferred ranges are from 1:3 to 1:300, more preferably from 1:10 to 1:100.

A combination may be present in the same or separate dosage forms, which may be administered simultaneously or sequentially. The composition may be suitable for oral administration with immediate and/or extended and/or sustained release to achieve systemic exposure to nicotinamide by delivering it to the circulation. Preferably, the composition according to the invention may be suitable for delayed release and/or delayed-controlled release of nicotinamide for specific local or topical efficacy in the lower small intestine and/or colon.

As used herein, the terms “preferred” or “preferably” refer to embodiments that may have certain benefits under certain circumstances, but other embodiments may also be preferred under the same or other circumstances. The recitation of one or more preferred embodiments does not imply exclusion of other useful embodiments from the scope of the invention. Terms like “comprises” and variations thereof do not have a limiting meaning in the description and claims. Citation of certain sections of documents from the literature does not imply that the rest of such documents is not relevant or not incorporated by reference. The recitations of numerical ranges by one or two endpoints includes all numbers subsumed within that range (e.g., “1 to 10” includes 1, 2.4, 4.576, etc., and “lower than 1” includes all numbers smaller than 1). For any method disclosed or cited herein that includes discrete steps, the steps may be conducted in any feasible order, and any combination of two or more steps may be conducted simultaneously. Any example or list of examples should not be interpreted as a restriction of any kind or as an exclusive list.

As used herein, the term “supplementation” refers to dietary supplementation of nicotinamide in patients with an infection (especially COVID-19). As used herein, the terms “treatment,” “treat,” and “treating” refer to reversing, alleviating, or inhibiting the progress of the disease resulting from the infection (especially COVID-19 and/or PCS), or one or more symptoms thereof, by administration of nicotinamide, as described herein. Preferably, supplementation or treatment may be administered after one or more symptoms have developed, alternatively preferred supplementation or treatment may be administered in the absence of symptoms. For example, supplementation or treatment may be administered to an individual prior to infection (pre-exposure prophylaxis) or an infected individual prior to the onset of symptoms (post-exposure prophylaxis). Supplementation or treatment may also be continued after symptoms have resolved, for example to prevent or delay their recurrence.

As used herein, the “lower small intestine” is the second half of the small intestine comprising the second half of the jejunum and the ileum. As used herein, the “terminal ileum” is the second half of the ileum.

As used herein, the term “topical efficacy” refers to a topical effect, in the pharmacodynamic sense, and thus refers to a local, rather than systemic, target for a dietary supplementation or medication. Accordingly, “local supplementation or efficacy” means a local supplementation or therapy with nicotinamide released specifically or selectively at a location where, for example, the dietary supplement or medication shall deliver its direct effect and nicotinamide enters the circulation to a lower degree than from conventional formulations with immediate and/or extended and/or sustained release, e.g., thereby causing only a reduced or low systemic action compared to conventional formulations. In this regard, the topical efficacy of the present invention is also contrasted with enteral (in the digestive tract) and intravascular/intravenous (injected into the circulatory system) administrations. In comparison to compositions aiming at high systemic availability and/or exposure, the at least partially topical efficacy of compositions may also be characterized by longer latency times until systemic levels of nicotinamide increase. Such latency times for topical release can be correlated with intestinal transit times known in the art (see, e.g., Davis et al. 1986, Gut 27:886; Evans et al. 1988, Gut 29:1035; Kararli 1995, Biopharm. Drug Dispos. 16:351; Sutton 2004, Adv. Drug Deliv. Rev. 56:1383). For example, after a variable time for gastric emptying (depending on the dosage form and feeding status and ranging from <15 minutes to more than 10 hours), small intestinal transit times are rather constant with 1-6 (usually 2-4) hours across formulations and studies (Davis et al. 1986, Gut 27:886). Thus, in case of doubt, the latency time in a fasted patient would usually be at least 1 hour for formulations with topical efficacy, at which time a formulation reaches the lower small intestine and systemic levels of nicotinamide may start to rise strongly. In the context of the present invention, topical efficacy can also be expressed in terms of a reduction of the plasma peak levels of at least 20%, 30%, 40%, 50%, 60%, 70%, 80%, 90% or even 95% or more relative to the same amount of nicotinamide administered in immediate-release formulations (e.g., nicotinamide in a capsule that dissolves in the stomach) in the same way and under the same conditions. As baseline values may differ strongly also within the same person, it is preferred to refer the peak levels to the respective baseline level immediately before the administration. Preferably, average plasma levels of a suitable cohort of persons are used for this definition of topical efficacy rather than the respective levels of single persons, which can yield highly divergent results (Schwarz et al. 2017, PCT/EP2017/058733). Topical efficacy is achieved in particular by the composition according to the invention as described herein. In combination formulations comprising nicotinamide formulations for immediate release and/or extended release and/or sustained release delivering nicotinamide mainly systemically to the circulation together with one or more nicotinamide formulations for delayed release and/or delayed-controlled release delivering nicotinamide mainly topically to the lower small intestine and/or colon, the above reduction of peak levels applies only to the delayed-release or delayed-controlled-release formulation, respectively.

It has previously been demonstrated that nicotinamide has a surprising anti-inflammatory effect by influencing the intestinal microbiota (the entirety of all microorganisms in the intestines, in particular the bacteria), which are mainly located in the lower small intestine and in the colon (Wätzig & Seegert 2013, PCT/EP2013/062363; Wätzig & Seegert 2015, PCT/EP2014/077637; Wätzig & Seegert 2015, PCT/EP2014/077646). The mechanism behind this surprising effect has been shown to involve nicotinamide-induced changes in the secretion pattern of antimicrobial peptides in the intestines, which supports the maintenance and/or regeneration of the normal, healthy intestinal microbiota (Hashimoto et al. 2012, Nature 487:477).

Therefore, as used herein, “beneficially influencing the intestinal microbiota” refers to causing a change in the intestinal microbiota that has a beneficial impact on health, especially on one or more of the diseases and conditions described herein, and/or to maintaining the healthy intestinal microbiota in preventive settings. For example, beneficial impacts may be associated with reducing the number of pathogenic bacteria, reducing the ratio of pathogenic bacteria to beneficial bacteria, increasing the diversity of the microbiota, increasing the amount of beneficial bacteria, partly or completely reverting pathological changes in the enterotype of the microbiota (e.g., enterotypes associated with Bacteroides, Prevotella and Ruminococcus), maintaining the healthy endogenous microbiota, and/or maintaining or improving or restoring metabolic pathways and/or their balance in the intestinal microbiota.

Thus, preferred according to the invention is a composition according to the invention for oral administration with at least partially delayed and/or delayed-controlled release of the active substance (preferably nicotinamide) for specific local supplementation or efficacy in the lower small intestine and/or the colon. More preferred, the composition is formulated for oral administration with at least partially delayed release of the active substance for specific local supplementation or efficacy in the lower small intestine and/or the colon. In another more preferred variant, the composition is formulated for oral administration with at least partially delayed-controlled release of nicotinamide for specific local supplementation or efficacy in the lower small intestine and/or the colon.

Preferably, nicotinamide is used in a dietary and/or pharmacological formulation that protects at least part of the nicotinamide from being absorbed by the body, e.g., from being absorbed into the circulatory system, in the stomach and/or upper small intestine and rather effects an at least partially topical release (e.g., delayed release and/or delayed-controlled release) into the lower small intestine and/or colon.

In particular, nicotinamide and the formulations and compositions described herein are thus suitable for being used in medicaments, medical products, nutraceuticals, foods for special medical purposes, dietary supplements, food ingredients and/or foods with at least partially topical release (e.g., delayed release and/or delayed-controlled release) to also enable direct nicotinamide supplementation or treatment of the lower small intestine and/or the colon, e.g., in the context of COVID-19 and/or PCS and its gastrointestinal symptoms, and/or a prolonged resorption period due to the continuous intestinal exposure.

Nicotinamide and the formulations and compositions described herein are equally usable in infections in both human and other mammals, in particular in domestic and useful animals. Examples of such animals are dogs, cats, minks, horses, camels, pigs or cows without objective restriction.

Nicotinamide may be used in any form available on the market in suitable nutritional or pharmaceutical quality, e.g., provided by general manufacturers and vendors like DSM, Lonza or Merck.

Preferably, the composition according to the invention contains a combination of two or more formulation variants of active substances, preferably of nicotinamide, in the same dosage form.

Preferably, the present invention also relates to combination preparations and/or compositions of nicotinamide, such as a variable dose combination or a fixed dose combination of immediate-release, sustained-release, extended-release, delayed-release and/or delayed-controlled-release nicotinamide. The different release kinetics of such formulations may be used to tailor the extent, duration and kinetics of systemic exposure and topical intestinal exposure to nicotinamide. The combinations described herein may be present in the same or separate dosage forms, which may be administered simultaneously or sequentially. The composition and dosage of such combinations is known to a person skilled in the art.

As used herein, the term “variable dose combination” refers to a combination of two or more formulation variants of nicotinamide in medicaments, medical products, nutraceuticals, foods for special medical purposes, dietary supplements, food ingredients and/or foods, whereby each formulation variant of nicotinamide is applied in the form of a separate composition, e.g., two single dosage forms. The separate compositions may be administered simultaneously, sequentially or on separate occasions by an administration regimen. For example, a composition of immediate-release nicotinamide (to be quickly absorbed after entering the stomach) in any suitable dosage thereof may be administered together, consecutively or subsequently, with a separate composition of delayed-release nicotinamide (to be partly or completely protected from absorption until reaching the lowed small intestine) in any suitable dosage thereof. Thus, variable dosages of two or more different formulations of nicotinamide may be combined. These variable dose combinations may use conventionally available compositions of medicaments, medical products, nutraceuticals, foods for special medical purposes, dietary supplements, food ingredients and/or foods or may be also achieved by customized polypharmacy via compounding. Depending on the presence and severity of gastrointestinal symptoms of a patient and/or expected benefits from a prolonged resorption period due to the continuous intestinal exposure, immediate-release, sustained-release, extended-release or controlled-release formulations for mainly systemic delivery and delayed-release or delayed-controlled-release formulations for mainly topical intestinal delivery may be administered in different proportions and dosages.

In contrast to a variable dose combination, a “fixed-dose combination” as used herein is a combination which is a formulation including two or more formulation variants of nicotinamide either combined in a single dosage form, which is manufactured and distributed in certain respective fixed doses, or in a combination of two or more separate dosage forms representing formulation variants of which a fixed number or amount is to be supplemented or administered according to label. A fixed-dose combination mostly refers to a mass-produced product having a predetermined combination and respective dosages.

The total dosage of the active substance (preferably nicotinamide) used according to the invention can be in the range of from 1 to 5000 mg, which may be administered as an individual dosage or as multiple dosages and/or a once, twice or more often daily dosage. The preferred total dosage of the active substance according to the invention is in the range of from 10 to 4000 mg, more preferably in the range of from 100 to 3000 mg.

As a non-limiting example, a high dose formulation can comprise up to 5000 mg of the active substance. For example, but not limited to, a high dose formulation can comprise a total of the active substance in the range of 1000-5000 mg, preferably in the range of 1000-4000 mg, more preferably in the range of 1000-3000 mg, e.g., 2000 mg.

As a non-limiting example, a low dose formulation can comprise up to 1000 mg, and preferably in a range of 1-1000 mg of the active substance, more preferably in a range of 100-1000 mg, of the active substance.

As a non-limiting example, a standard dose formulation can comprise up to 3000 mg, and preferably in a range of 250-2500 mg, more preferably in a range of 500-2000 mg, of the active substance.

A non-limiting particular example of a fixed-dose high dose formulation comprises a combination of 1000 mg immediate-release nicotinamide and 1000 mg delayed-release and/or delayed-controlled-release nicotinamide.

A non-limiting particular example of a fixed-dose standard dose formulation comprises a combination of 750 mg immediate-release nicotinamide and 750 mg delayed-release and/or delayed-controlled-release nicotinamide.

A non-limiting particular example of a fixed-dose low dose formulation comprises a combination of 400 mg or 500 mg immediate-release nicotinamide and 400 or 500 mg delayed-release and/or delayed-controlled-release nicotinamide.

Such compositions of the invention may, for example, preferably be administered as tablets, pellets or granulates, preferably microgranulates, if suitable in a capsule, sachet or stick pack, and preferably in a sachet or stick pack.

It is preferred that the active substance (preferably nicotinamide) is formulated in the form of tablets, granules, microgranules or pellets. These tablets, granules, microgranules or pellets can be used for single dosage forms or for variable dose combinations or fixed dose combinations. If different formulation variants of the active substance in the form of tablets, granules, microgranules or pellets are used as described herein, these may be used in the form of any single dietary or pharmaceutical composition, as well as a variable dose combination or a fixed dose combination. Granules, microgranules or pellets may be compressed into tablets, or filled into capsules, sachets or stick packs, or used as such, as appropriate.

In order to produce orally administered formulations of the active substance (e.g., tablets, dragees, capsules, sachets, etc.) for at least partial release in the lower small intestine and/or in the colon, it is advantageous to use delayed modes of release. In contrast to conventional (in some cases also delayed, but systemically delivering) modes of release for optimum supplementation of certain embodiments of the present invention, e.g., immediate-release, extended-release and/or sustained-release nicotinamide formulations, such delayed or and/or delayed-controlled modes of release (at least) partially or (even) substantially avoid an absorption in the stomach and in the upper portions of the small intestine.

For oral administration, particular dosage forms that at least partially control and/or delay the release of the active substance due to special galenics are particularly suitable. Such dosage forms may be simple tablets and also coated tablets, e.g., film tablets or dragees. The tablets are usually oblong, round or biconvex. Particular oblong tablet forms, which allow the tablet to be separated, can be preferred. In addition, minitablets, granules, spheroids, pellets or microcapsules are possible (e.g., Liang & Dingari 2017, PCT/US2017/028063; Schwarz et al. 2017, PCT/EP2017/058733), which are filled into capsules, sachets or stick packs, where appropriate. In order to deliver nicotinamide in part in a systemically acting formulation (e.g., immediate release) and in a formulation acting largely topically on the lower small intestine and/or colon (e.g., delayed and/or delayed-controlled release), combinations of different formulations in separate dosage forms and/or multilayer dosage forms can be used to first release part of the the active substance in the stomach and upper small intestine and release the other part from, e.g., a quickly disintegrating core (delayed release) or a matrix core (delayed-controlled release) with or without pH-dependent or microbial-dependent release. Another example are erosion-based release technologies exemplified by the OralogiK™ product portfolio (BOO Pharma).

The term “delayed release” relates preferably to a formulation or component thereof that releases, or delivers, the active substance after a period of delay, e.g., degradation of a film coating or other coating due to the pH, chemical, enzymatic and/or microbial environment that is preferably present in the lower small intestine and/or colon. In certain embodiments, the delay is sufficient for at least a portion of the active substance in a formulation to be released in the lower small intestine and/or colon.

The term “delayed-controlled release” refers preferably to a formulation or component thereof that releases, or delivers, the active substance over a prolonged period of time (time-dependent release) and/or under certain physiological conditions, e.g., degradation of a coating or matrix due to the pH, chemical, enzymatic and/or microbial environment that is preferably present in the lower small intestine and/or colon. In certain embodiments, the period of time or the release according to physiological conditions is sufficient for at least a portion of the nicotinamide in a formulation to be released in the lower small intestine and/or colon.

The retardation and/or delayed release and/or delayed-controlled release is advantageously achieved, e.g., by coatings which are resistant to gastric juice and dissolve depending on the pH, by using different carrier matrix components (e.g., different grades of hydroxypropyl cellulose, hydroxypropyl methylcellulose, microcrystalline cellulose, sodium carboxymethylcellulose, starch, modified starch, pregelatinized starch, gelatin, polyvinylpyrrolidone) or combinations thereof, by means of other matrix and/or multi-matrix (MMX) technologies, or a combination of these techniques. Examples include film coatings which contain acrylic and/or methacrylate polymers in various mixtures for delayed release.

Additional examples include biodegradable polymers like natural or chemically modified polymers and polymer-drug conjugates, coatings and/or matrix agents for microbiota-dependent release (reviewed, e.g., by Rajpurohit et al. 2010, Indian J. Pharm. Sci. 72:689). For example, the active substance can be contained in a matrix comprising components as described above, which is coated with a material that provides the delayed release of the active substance. The active substance according to the invention can be administered in, e.g., tablets, minitablets, granules, spheroids, pellets, microcapsules or large-volume capsules (e.g., gelatin or hydroxypropyl methylcellulose capsules), which are coated by means of known methods. Suitable coating agents are water-insoluble waxes, such as carnauba wax, and/or polymers, such as poly(meth)acrylates, e.g., the entire poly(meth)acrylate product portfolios with the trade names Eudraguard® and Eudragit® provided by from Evonik Industries, in particular Eudraguard® protect, Eudraguard® control, Eudraguard® biotic, Eudraguard® natural, Eudragit® L 30 D-55 (an aqueous dispersion of anionic polymers with methacrylic acid as a functional group), Eudragit® L 100-55 (which contains an anionic copolymer based on methacrylic acid and ethyl acrylate), Eudragit® L 100 or L 12.5 or S 100 or S 12.5 (anionic copolymers based on methacrylic acid and methyl methacrylate), combinations of Eudragit® S and L compounds, or Eudragit® FS 30 D (an aqueous dispersion of an anionic copolymer based on methyl acrylate, methyl methacrylate and methacrylic acid), and/or water-insoluble celluloses (e.g., methyl cellulose or ethyl cellulose). Where appropriate, water-soluble polymers (e.g., polyvinylpyrrolidone), water-soluble celluloses (e.g., hydroxypropylmethyl cellulose or hydroxypropyl cellulose), emulsifiers and stabilisers (e.g., polysorbate 80), polyethylene glycol (PEG), lactose or mannitol can also be contained in the coating material.

In preferred embodiments, formulations for immediate release and/or extended release and/or sustained release are equipped with taste-masking technologies comprising, alone or in combination, e.g.,
organoleptic methods, such as adding combinations of sweeteners (e.g., sucralose, aspartame, acesulfame potassium, glycerrhizin, cyclamate, lactose, mannitol, saccharin, or sucrose), sugars, flavours (e.g., mint, peppermint, menthol, wild cherry, walnut, chocolate, passion fruit or citrus flavours like lemon or orange), bitterness-blocking agents (e.g., adenosine monophosphate, dihydrochalone, sodium chloride, sodium acetate, sodium gluconate, lipoproteins [e.g., composed of phosphatidic acid and β-lactoglobulin] or phospholipids [e.g., phosphatidic acid, phosphatidylinositol or soy lecithin]), effervescent agents (e.g., generators of carbon dioxide), taste-modifiers, buffers and/or other excipients (e.g., zinc sulfate, maltodextrins [e.g., pea maltodextrin] or polyols) to the composition or a coating thereof;
single- or multi-layer coatings and/or matrix technologies comprising, alone or in combination, e.g., hydrophobic or hydrophilic polymers (e.g., methacrylic acid and methacrylic ester copolymers like Eudragit® E, E-100, RL 300, RS 300, L30D-55 or NE 30D; Eudraguard® protect, natural or control; ethylcellulose; hydroxypropylmethylcellulose; hydroxypropylcellulose; cellulose acetate; croscarmellose; polyvinyl alcohol; polyvinylpyrrolidone (e.g., PVP-K30 or Kollicoat); polyvinyl acetate; shellac; guar gum), lipids (e.g., glyceryl palmitostearate, glyceryl monostearate or glycerol behenate), talc, detergents (e.g., sodium lauryl sulfate or polysorbates like polysorbate 80), sugars and/or sweeteners (see above);
matrix granulation (e.g., with gelling or lipid polymers);
solid dispersions using melting, solvent or melting-solvent methods and carriers like povidone, polyethylene glycols of various molecular weights, hydroxypropylmethylcellulose, urea, mannitol or ethylcellulose; spray-drying (e.g., with ethylcellulose, hydroxypropylmethylcellulose; hydroxypropylcellulose or acrylate polymers); hot-melt extrusion (e.g., with ethylcellulose, hydroxypropylmethylcellulose; hydroxypropylcellulose or acrylate polymers), preferably followed by milling or micronizing of the extrudates to obtain taste-masked granules or particles, which are preferably subsequently incorporated into a suitable dosage form;
microencapsulation using the coating agents listed above (e.g. Wurster fluid bed coating [e.g., with croscarmellose, Eudragit]);
barrier membranes;
inclusion complexation (e.g., by cyclodextrins, tannic acid, Eudragit® polymers like Eudragit S-100 or chitosan);
ion exchange resins, such as copolymers of styrene, acrylic acid or methacrylic acid with divinylbenzene;
adsorption (e.g. using silicates, silica gel or bentonite).
Further non-limiting examples especially for the formulation of dietary supplements, but also food ingredients, according to the present invention have been described using maltodextrin-pectin microcapsules and shellac-coated granulates (Berg et al. 2012, J. Food Eng. 108:158; Schwarz et al. 2017, PCT/EP2017/058733; Theismann et al. 2019, Int. J. Pharm. 564:472).

The composition, e.g., a formulation of medicaments, medical products, nutraceuticals, foods for special medical purposes, dietary supplements, food ingredients and/or foods, can also contain further excipient substances, such as binders (e.g., methylcellulose, carboxymethylcellulose sodium, hydroxypropyl cellulose, hydroxypropyl methylcellulose/hypromellose, hydroxyethyl cellulose, dextrin, maltodextrin, copovidone and/or sodium alginate), fillers (e.g., anhydrous lactose, lactose monohydrate, starch, pregelatinized starch, powdered cellulose, calcium carbonate, magnesium carbonate, anhydrous dibasic calcium phosphate, dibasic calcium phosphate dihydrate, anhydrous calcium sulfate, calcium sulfate dihydrate, tribasic calcium phosphate, sucrose, fructose, anhydrous glucose/dextrose, glucose/dextrose monohydrate, sorbitol, mannitol, maltitol, isomalt and/or xylitol), glidants, lubricants and flow regulating agents. The nicotinamide according to the invention can be formulated, where appropriate, together with further active substances and with excipients conventional in dietary or pharmaceutical compositions, e.g., talcum, gum arabic, lactose, starch, magnesium stearate, cocoa butter, aqueous and non-aqueous carriers, lipid components of animal or vegetable origin, paraffin derivatives, glycols (in particular polyethylene glycol), various plasticizers, dispersants, emulsifiers and/or preservatives.

According to the invention, a composition is preferred, wherein part of the active substance(s), preferably nicotinamide, is formulated for delayed or delayed-controlled release in order to enter the circulatory system only to a low degree, so that plasma peak levels of active substance(s), preferably nicotinamide, following administration of the delayed or delayed-controlled release formulation are reduced by at least 20%, 30%, 40%, 50%, 60%, 70%, 80%, 90%, or 95% relative to the same amount of active substance(s), preferably nicotinamide, administered in immediate-release formulations in the same way and under the same conditions.

A further aspect of the invention described herein is the efficient use of the described medicaments, medical products, nutraceuticals, foods for special medical purposes, dietary supplements, food ingredients and/or foods on the basis of blood and/or urine and/or stool and/or genetic and/or microbiological and/or other biomarkers or data and specific needs of the individuals to be treated. In particular, serum levels of e.g., tryptophan, nicotinamide and their metabolites can be used to direct supplementation or therapeutic decisions. Evidence-based personalized medicine including analyses and data of the disease course, the pathogen variant or strain and/or the genetic background (e.g., genes coding for cell surface receptors, transporter proteins, metabolism enzymes or signal transduction proteins, which interact with the pathogen, the immune response to the pathogen, nicotinamide and/or its metabolites and/or its downstream effectors) can contribute information and improvements with respect to the type of use, the mode of application, the time(s) of use, the dose and/or the dosage regimen of the medicaments, medical products, nutraceuticals, foods for special medical purposes, dietary supplements, food ingredients and/or foods described herein. Individuals who may benefit from this personalised treatment include those with disease-specific or non-specific changes in blood and/or plasma and/or serum lipids and/or other biomarkers. This applies analogously to analyses of the intestinal microbiota, particularly when a stool sample indicates a change in the microbiota. The present invention thus also comprises the use of suitable test methods to identify individuals particularly susceptible to the medicaments, medical products, nutraceuticals, foods for special medical purposes, dietary supplements, food ingredients and/or foods according to the invention and/or to adapt the use of these as well as concomitant supplementation and/or medication to the individual circumstances. This also comprises expressly the use of different formulation variants or compositions comprising the active substance or combinations thereof in different modes of administration depending on the biomarkers of the individual to be supplemented or treated. For these purposes, it is possible to use laboratory tests and/or suitable test kits and also measuring methods, devices and/or kits to be employed by a physician, user and/or patient, e.g., to analyze suitable parameters in the blood, urine or other body fluids or in stool samples. In particular, the present invention also relates to using these biomarkers to support patient or subject selection for the supplementation or treatment described herein, to personalise and adapt the compositions and/or supplementations and/or treatments described herein, and/or to determine end points and efficacy benchmarks for the compositions and/or supplementations and/or treatments described herein.

It is preferred according to the invention that the composition according to the invention is formulated for use for administration once daily. Surprisingly, this regimen of administration showed superior effects (Wätzig & Schreiber 2021, PCT/EP2021/083138). The best effects were gained when the composition according to the invention was administered with breakfast in the morning. For dosing once daily, a nicotinamide content of 1 to 5000 mg per finished dosage form, preferably 10 to 4000 mg and more preferably 100 to 3000 mg is preferred.

It is also preferred that compositions according to the invention are combined with foods, beverages, dietary supplements, foods for special medical purposes, probiotics, prebiotics, synbiotics and/or vitamins, especially with those that are intended to support the recovery from post-acute symptoms of an infection. Probiotics are ingestible live microbial cultures, which survive transit through the gastrointestinal tract and beneficially affect the host by improving its intestinal microbial balance. Prebiotics are non-digestible and selectively fermented food ingredients or supplements that allow specific changes in the composition and/or activity of the gastrointestinal microbiota which are beneficial for host well-being and health. Examples for prebiotics are resistant starch, fructo-oligosaccharides, galacto-oligosaccharides, xylooligosaccharides, polydextrose, lactulose, inulin or soluble fibre (e.g., psyllium husk or acacia fibres). Synbiotics are a combination of pro- and prebiotics.

As described above and can be seen from the examples, the composition according to the invention was very effective in preventing or reducing one or more symptoms of PCS, preferably impaired sense of smell and/or taste. Accordingly, a composition according to the invention is preferred for use for preventing or reducing one or more post-acute symptoms of the infection, wherein the at least one post-acute symptom is selected from the group consisting of reduced physical performance, fatigue, exercise intolerance, chemosensory deficits, joint pain, muscle pain, chest pain, ear-nose-throat ailments, cough, wheezing, gastrointestinal ailments, neurological ailments, psychological ailments, dermatological ailments, infection signs, and sleep disturbance, wherein the particularly preferred selected symptoms are impaired sense of smell and/or taste preferably at 6 months or later after patients tested positive for the infection.

Preferably, this effect is achieved at least for 10%, preferably 15%, more preferably 20%, even more preferably 25% and most preferably 30% more of those patients compared to placebo. It is alternatively or additionally preferred that the effect is achieved in a similar percentage of male and female patients, wherein similar means +/−25%, preferably +/−10%.

The composition according to the invention is preferably used for administration to patients with at least one characteristic or underlying medical condition associated with an increased risk of developing severe illness from the infection (especially COVID-19) and/or post-acute symptoms (especially PCS) (Wätzig & Schreiber 2021, PCT/EP2021/083138).

Alternatively or in addition to that, a composition according to the invention is preferred for a use wherein treatment and/or supplementation is administered to patients with at least one characteristic symptom of the infection. For acute COVID-19, such symptoms are preferably selected from the group consisting of cough, fever, impaired sense of taste and taste loss.

### EXEMPLIFICATION

There are variable possibilities to advantageously develop, and develop further, the teaching of the present invention. For this purpose, reference is made to the examples below which describe the invention in a representative way.

### Example 1: Formulation and Product Variants (General Information for Performing the Invention)

(1) Nicotinamide or suitable precursors or metabolites thereof can be administered alone or in combination in a tablet or other solid dosage forms such as, e.g., granules, pellets or microcapsules as described in the present application with a core comprising the active substance (preferably nicotinamide) in a matrix for at least partially controlled release (e.g., comprising different grades of hydroxypropyl cellulose, hydroxypropyl methylcellulose, microcrystalline cellulose, sodium carboxymethylcellulose, starch, modified starch, pregelatinized starch, gelatin, polyvinylpyrrolidone, or combinations thereof, or employing other matrix or multi-matrix technologies).

(2) The core of Example 1 (1) can be provided with a coating for delayed release comprising a film coating which contains acrylic and/or methacrylate polymers in various mixtures for delayed release or biodegradable polymers for microbiota-dependent release. Further suitable coating agents are water-insoluble waxes, such as carnauba wax, and/or polymers, such as poly(meth)acrylates, e.g., the entire poly(meth)acrylate product portfolios with the trade names Eudraguard® and Eudragit® provided by from Evonik Industries, in particular Eudraguard® protect, Eudraguard® control, Eudraguard® biotic, Eudraguard® natural, Eudragit® L 30 D-55 (an aqueous dispersion of anionic polymers with methacrylic acid as a functional group), Eudragit® L 100-55 (which contains an anionic copolymer based on methacrylic acid and ethyl acrylate), Eudragit® L 100 or L 12.5 or S 100 or S 12.5 (anionic copolymers based on methacrylic acid and methyl methacrylate), combinations of Eudragit® S and L compounds, or Eudragit® FS 30 D (an aqueous dispersion of an anionic copolymer based on methyl acrylate, methyl methacrylate and methacrylic acid), and/or water-insoluble celluloses (e.g., methyl cellulose, ethyl cellulose). Where appropriate, water soluble polymers (e.g., polyvinylpyrrolidone), water-soluble celluloses (e.g., hydroxypropylmethyl cellulose or hydroxypropyl cellulose), emulsifiers and stabilisers (e.g., polysorbate 80), polyethylene glycol (PEG), lactose or mannitol are also contained in the coating material.

(3) The core of Example 1 (1) can be provided with taste-masking technologies as described in the Detailed Description which can comprise single- or multi-layer coatings comprising, alone or in combination, e.g., hydrophobic or hydrophilic polymers (e.g., methacrylic acid and methacrylic ester copolymers like Eudragit® E, E-100, RL 30D, RS 30D, L30D-55 or NE 30D; Eudraguard® protect, natural or control; ethylcellulose; hydroxypropylmethylcellulose; hydroxypropylcellulose; cellulose acetate; croscarmellose; polyvinyl alcohol; polyvinylpyrrolidone (e.g., PVP-K30 or Kollicoat); polyvinyl acetate; shellac; guar gum), lipids (e.g., glyceryl palmitostearate, glyceryl monostearate or glycerol behenate), talc, detergents (e.g., sodium lauryl sulfate or polysorbates like polysorbate 80), sugars and/or sweeteners.

(4) The core of Example 1 (1) can be provided with a coating layer system comprising an inner layer for delayed release in the lower small intestine and/or colon according to Example 1 (2), a layer of active substance formulated for immediate-release and an outer layer comprising taste-masking technologies as described in Example 1 (3).

(5) Nicotinamide or suitable precursors or metabolites thereof can be administered alone or in combination in a tablet or other solid dosage forms as described herein based on or analogous to the OralogiK technology (BOO Pharma) or the Geoclock technology (Skyepharma), with an immediate-release, delay and later pulse release kinetic as described by the manufacturers.

(6) Nicotinamide or suitable precursors or metabolites thereof can be granulated alone or in combination in an immediate-release pellet, minipellet or micropellet formulation, of which a fixed or variable part can be provided with a coating that effects delayed (e.g. pH-dependent) release and the other part may optionally be provided with a taste-masking coating for immediate release [for details, see Examples 1 (1), (2) and (3)]. The two types of pellets or pellets with combined immediate- and controlled and/or delayed release properties as described herein, can be filled together into a single sachet, stick pack, seal lid, bottle lid, capsule or other suitable container, preferably in a fixed proportion, e.g. 2:1 or 1:1 for immediate:delayed release. Alternatively, the two types of pellets can be filled into separate sachets, stick packs or capsules and are administered depending on the symptoms of the patient. In further alternatives, the pellets can be filled into capsules or incorporated into tablets.

(7) Nicotinamide or suitable precursors or metabolites thereof can be administered alone or in combination in a tablet or other solid dosage forms as described herein with a core comprising the active substance (preferably nicotinamide). The core can have no matrix properties for controlled release and can be equipped with taste-masking technologies as described in Example 1 (6).

(8) Nicotinamide or suitable precursors or metabolites thereof can be administered alone or in combination in a tablet or other solid dosage forms as described herein that are combined with foods, beverages, dietary supplements, foods for special medical purposes, probiotics, prebiotics, synbiotics and/or vitamins. In a preferred example, pellets are administered together with a beverage or (semi)liquid matrix (e.g., a fruit smoothie or a dairy product like liquid yoghurt). In a further preferred example, the beverage or (semi)liquid matrix is slightly acidic to prevent disintegration of pH-dependent delayed-release coatings. In a further preferred embodiment, solid dosage forms of the invention are administered together with solid or liquid foods for special medical purposes.

(9) Nicotinamide, nicotinic acid and tryptophan can be administered in combination in a tablet or other solid dosage forms as described herein. In a preferred embodiment, such compositions are suitable as medicaments or foods for special medical purposes for conditions with enhanced requirements for NAD precursors. In a further preferred embodiment suitable as a dietary supplement or food for special medical purposes, the total amount of niacin equivalents contained in the formulation does not exceed a total of 160 mg. For example, 150 mg nicotinamide, 4 mg nicotinic acid and 200 mg tryptophan (3,333 niacin equivalents) are combined, wherein these doses reflect several national European recommendations for the addition of said active substances to foods including dietary supplements.

### Example 2: Results from the Pilot Phase of the COVit-2 Dietary Intervention Trial in COVID-19 Patients with Mild to Moderate Disease

In the monocentric, randomized, double-blind, prospective, placebo-controlled dietary intervention trial COVit-2 (DRKS00021214; NCT04751604) conducted at the Department of Internal Medicine I of the University Medical Center Schleswig-Holstein, Campus Kiel (Germany), outpatients with early symptomatic COVID-19 (up to 7 days after their first positive SARS-CoV-2 PCR test) in domestic quarantine were recruited from February 2021 to January 2022. As the vaccinations against SARS-CoV-2 became available to the broader public only after several hundred patients had already been recruited into the COVit-2 trial, partly or fully vaccinated patients were excluded from participation to maintain the same inclusion population and ensure formal comparability of all patients within the trial. However, there is no reason or evidence to assume that the fundamental metabolic and microbiota-targeting effects of nicotinamide could be any different in symptomatic COVID-19 patients with or without prior vaccination.

For details of the trial, see Wätzig & Schreiber 2021, PCT/EP2021/083138. Patients were randomized to oral self-administration (once daily with breakfast for 28 days) of either a combination of two different nicotinamide tablets (one conventional 500-mg immediate-release nicotinamide tablet and one novel 500-mg controlled-ileocolonic-release nicotinamide [CICR-NAM]tablet, which ensures prolonged and continuous intestinal exposure to nicotinamide) or matching placebo tablets. The entire trial was conducted remotely. Patients registered online and were contacted by the study team to check their eligibility and to supply the written patient information and informed consent forms. After informed consent, patients were called for baseline data acquisition (week 0) and subsequently at week 2, week 4 and week 6. The queried baseline information included personal and demographic data, smoking status, comorbidities, concomitant administration of dietary supplements or medicaments as well as COVID-19 symptoms. At weeks 2, 4 and 6, regular intake of the trial supplements and concomitant supplements and medicaments, respectively, was queried as well as the current COVID-19 symptoms and disease course.

Approximately 6 months after infection, patients were contacted for follow-up interviews and a large battery of questionnaires and tests including antibody level measurements. In addition to the patient interviews and SF-36 (quality of life) and FACIT-F (fatigue) questionnaires also used during the first 6 weeks after the beginning of the dietary intervention (Wätzig & Schreiber 2021, PCT/EP2021/083138), the 6-month test battery particularly focused on PCS symptoms. A validated smell test (University of Pennsylvania Smell Identification Test™; Sensonics/MediSense) was performed by the patient and recorded as patient-reported outcome by a paper questionnaire. Questionnaires on olfactory and gustatory abilities (incl. the Self-reported Mini Olfactory Questionnaire, Self-MOQ, and the Questionnaire of Olfactory Disorders, QOD), respiration (Multidimensional Dyspnea Profile, MDP), mental state (Patient Health Questionnaire Depression, PHQ-8; Generalised Anxiety Disorder 7, GAD-7; Perceived Stress Scale, PSS; Brief Resilience Scale, BRS), sleep quality (Pittsburgh Sleep Quality Index, PSQI) and fatigue (Multidimensional Fatigue Inventory, MFI) were recorded as patient-reported outcomes by paper questionnaires. In addition, a cognition test (Telephone Adaptation of the Modified Mini-Mental State Exam, T3MS) validated for telephone interviews was performed.

For the antibody level measurements, the time between the positive SARS-CoV-2 PCR test and the date of blood sampling for antibody analysis as well as, if applicable, the intervals between one or more subsequent vaccinations against SARS-CoV-2 and the blood sampling date were recorded. Antibody levels were measured using the AProof Duo Test (Adversis Pharma, now AP Diagnostics; Leipzig, Germany). In this test, dried blood spot samples are rehydrated and analysed by ELISAs detecting antibodies directed against the nucleocapsid (N) or spike (S) protein of SARS-CoV-2. Antibodies against the N protein are analysed to evaluate immunity generated only by SARS-CoV-2 infection. After infection with SARS-CoV-2, levels of antibodies against the S protein can represent both the consequences of infection (if no additional vaccination is performed) or of the combined humoral immunity generated by infection and subsequent vaccination(s). Between the 6-week observation period of the acute infection and the follow-up after at least 6 months, many trial participants had been vaccinated against SARS-CoV-2. Anti-N antibody levels were quantified as percent of the signal of a positive control consisting of an anti-N antibody pool from five donors collected at different time points starting 2 weeks after PCR-confirmed infection with SARS-CoV-2. This anti-N antibody pool was diluted in the negative control medium at a fixed ratio. The detection was considered negative if below 20% of the positive control, borderline for 20-30% and positive if above 30%. Anti-S antibody protein levels were quantified as binding antibody units per milliliter (BAU/mL; according to the WHO International Standard for anti-SARS-CoV-2 immunoglobulin [human][NIBSC code 20/136]). The detection was considered negative if below 22 BAU/mL, borderline for 22-44 BAU/mL, positive if above 44 BAU/mL and positive for neutralising antibodies at 100 BAU/mL or more.

For the first 50 patients of each trial arm (nicotinamide vs. placebo) with complete data for the respective analyses, data quality control tests were performed and surprisingly revealed that nicotinamide supplementation actually increased levels of both anti-N and anti-S antibodies (Table 1). Borderline values (2-3 per parameter and group of n=50 each) are not shown.

TABLE 1
Levels of anti-SARS-COV-2 antibodies

| | Anti-N antibodies | | Anti-S antibodies | |
|---|---|---|---|---|
| | [% + control) | | [BAU/mL] | |
| | Nicotinamide | Placebo | Nicotinamide | Placebo |
| Median | 10% | 6% | 813 | 671 |
| n positive | 14 | 9 | 47 | 44 |
| % positive | 28% | 18% | 94% | 88% |
| n negative | 33 | 39 | 1 | 3 |
| % negative | 66% | 78% | 2% | 6% |

Moreover, the difference in the median of anti-S antibodies was much stronger in patients with at least one characteristic or underlying medical condition associated with an increased risk of developing severe COVID-19, namely 1,121 BAU/mL (nicotinamide) vs. 692 BAU/mL (placebo).

Despite the small sample size and the partly moderate effect sizes, effects on diverse symptoms of PCS were already observed in this pilot population: patients supplemented with nicotinamide had
a median PSS score of 24 compared to 28 in the placebo group, indicating lower stress (Schneider et al. 2020, Int. J. Clin. Health Psychol. 20:173);
a median PHQ-8 score of 4 compared to 6 in the placebo group, indicating a lower tendency toward depression (Kroenke et al. 2009, J. Affect. Disord. 114:163);
a median GAD-7 score of 3 compared to 6 in the placebo group, indicating lower anxiety (Spitzer et al. 2006, Arch. Intern. Med. 166:1092);
a 2% proportion of patients with strong microsmia/hyposmia (UPSIT score <26) compared to 12% in the placebo group (Doly etaL. 1989, Percept. Psychophys. 45:381);
an 8% proportion of patients with mild cognitive impairment (T3MS score <89) compared to 12% in the placebo group (Alexopoulos et al. 2006, Fortschr. Neurol. Psychiatr. 74:329), with a slightly stronger signal in patients with at least one characteristic or underlying medical condition associated with an increased risk of developing severe COVID-19, namely 10% in the nicotinamide group vs. 21% in the placebo group.

Taken together, nicotinamide increased anti-SARS-CoV-2 antibody levels and reduced a plethora of key PCS symptoms in these first n=50 patients of the COVit-2 trial.

### Example 3: Final Results from the COVit-2 Dietary Intervention Trial in COVID-19 Patients with Mild to Moderate Disease

The COVit-2 trial (see Example 2) was expanded to a total of n=900 randomized patients. Of these, n=500 patients had at least one characteristic or underlying medical condition associated with an increased risk of developing severe illness from COVID-19, which was the population that had benefited most from nicotinamide supplementation in the acute phase of the disease (Wätzig & Schreiber 2021, PCT/EP2021/083138) and is hereinafter referred to as ‘risk factor population’.

Individual symptoms In the acute phase of COVID-19 from baseline (up to 7 days after testing positive for SARS-CoV-2) until Week 6, administration of nicotinamide conferred a biologically and statistically significant benefit with regard to different parameters of physical performance (mainly at week 2), as shown by in the interim analysis of n=402 patients of the COVit-2 trial (Wätzig & Schreiber 2021, PCT/EP2021/083138).

However, nicotinamide supplementation had no beneficial effect on the impaired sense of smell and/or taste experienced by some patients, even though preliminary data in the pilot trial COVit-1 had indicated a potential benefit (Wätzig & Schreiber 2021, PCT/EP2021/083138). In contrast, in the final analysis of n=500 patients of the risk factor population (the primary analysis population for acute COVID-19 in the COVit-2 trial), there was rather a trend favouring placebo, as evidenced by the rates of resolved, constant or newly occurring impairment shown in Table 2 to Table 5 below.

Here, the efficacy variables “impaired sense of smell” (yes/no) and “impaired sense of taste” (yes/no) from patient interviews represent the broadest and most sensitive approach, as other more specific tests or questions miss aspects of smell or taste that are not explicitly queried and thus may fail to detect significant differences. In the acute phase of the COVit-2 trial, these variables were analyzed using change to baseline, considered as resolution (symptom present at baseline but not at week x), persistence (no change compared to baseline) or worsening (symptom present at week x but not at baseline). The frequency of patients with symptom resolution, persistence or worsening for all weeks by treatment group was compared using the Cochran-Mantel-Haenszel test. Post-hoc tests for each week were calculated using the Fisher Exact test with Benjamini & Hochberg adjustment for multiple testing. The Woolfe test was performed to test for homogeneity of odds ratios across time. In case of significant p-values of the Woolfe test, the Cochran-Mantel-Haenszel test would not have been be appropriate.

TABLE 2
Impaired sense of smell in acute COVID-19:
frequencies of change and statistical tests

| Treatment | Change | Week 2 | Week 4 | Week 6 |
|---|---|---|---|---|
| Nicotinamide | Resolution of impairment (return to normal) | 61 | 78 | 92 |
| Nicotinamide | Persistence (no change) | 155 | 150 | 141 |
| Nicotinamide | Worsening (new impairment) | 32 | 20 | 15 |
| Placebo | Resolution of impairment (return to normal) | 67 | 95 | 100 |
| Placebo | Persistence (no change) | 160 | 138 | 134 |
| Placebo | Worsening (new impairment) | 25 | 19 | 18 |

| Test | Test statistic | P value |
|---|---|---|
| Cochran-Mantel-Haenszel test | Cochran-Mantel-Haenszel M\hat{ }2 2.299 | 0.317 |
| Woolfe test on Homogeneity of Odds | X-squared 0.19 | 0.909 |
| Ratios (no 3-way assoc.) | | |

TABLE 3
Impaired sense of smell in acute COVID-19: post
hoc comparison (Fisher Exact test) of the change
to baseline for the two treatment groups

| | Time | P value* |
|---|---|---|
| Nicotinamide vs. Placebo | 2 Weeks | 0.555 |
| Nicotinamide vs. Placebo | 4 Weeks | 0.344 |
| Nicotinamide vs. Placebo | 6 Weeks | 0.703 |

*Fisher Exact test.

TABLE 4
Impaired sense of taste in acute COVID-19:
frequencies of change and statistical tests

| Treatment | Change | Week 2 | Week 4 | Week 6 |
|---|---|---|---|---|
| Nicotinamide | Resolution of impairment | 56 | 74 | 83 |
| | (return to normal) | | | |
| Nicotinamide | Persistence (no change) | 170 | 159 | 157 |
| Nicotinamide | Worsening (new impairment) | 22 | 15 | 8 |
| Placebo | Resolution of impairment | 76 | 96 | 102 |
| | (return to normal) | | | |
| Placebo | Persistence (no change) | 157 | 145 | 141 |
| Placebo | Worsening (new impairment) | 19 | 11 | 9 |

| Test | Test statistic | P value |
|---|---|---|
| Cochran-Mantel-Haenszel test | Cochran-Mantel-Haenszel | 0.007 |
| | M\hat{ }2 10.045 | |
| Woolf-test on Homogeneity of Odds | X-squared 0.013 | 0.994 |
| Ratios (no 3-Way assoc.) | | |

TABLE 5
Impaired sense of taste in acute COVID-19: post
hoc comparison (Fisher Exact test) of the change
to baseline for the two treatment groups

| | Time | P value |
|---|---|---|
| Nicotinamide vs. Placebo | 2 Weeks | 0.155 |
| Nicotinamide vs. Placebo | 4 Weeks | 0.134 |
| Nicotinamide vs. Placebo | 6 Weeks | 0.250 |

*Fisher Exact test.

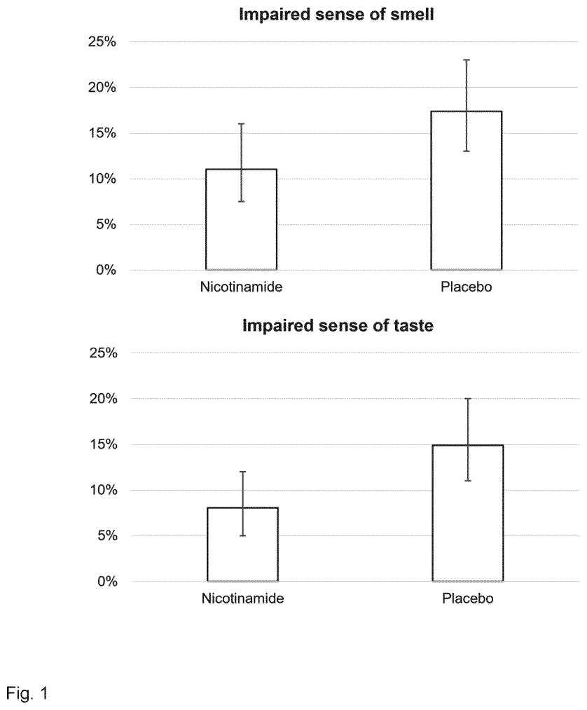

Surprisingly, nicotinamide supplementation led to significantly reduced frequencies of impaired sense of smell or taste after six months (Table 6 and FIG. 1), even though nicotinamide was clearly ineffective at influencing these symptoms in the acute phase of the disease.

TABLE 6
Impaired sense of smell and taste at 6 months after COVID-19

| | Nicotinamide | | Placebo | | P value³ |
|---|---|---|---|---|---|
| Characteristic | N = 236¹ | 95% CI² | N = 242 | 95% CI² | 3 |
| Impaired | 26 (11.02%) | [7.5%; 16% ] | 42 (17.36%) | [13%; 23% ] | 0.047 |
| sense of smell | | | | | |
| Impaired | 19 (8.05%) | [5.0%; 12% ] | 36 (14.88%) | [11%; 20% ] | 0.019 |
| sense of taste | | | | | |

¹n (%)
²CI = Confidence Interval
³Pearson's Chi-squared test; Fisher's exact test

When focusing on the per-protocol patients (n=235 for nicotinamide and n=237 for placebo), P values were further reduced to P=0.039 for impaired sense of smell and P=0.016 for impaired sense of taste, respectively. The per-protocol population included only those patients who did not drop out and who complied regarding investigational product intake for at least 80%, i.e. intake of investigational product for at least 11 of 14 days between each trial interval in the acute phase (week 0-week 2 and week 2-week 4).

### PCS Score

In agreement with an overall mild to moderate disease course, most patients in the COVit trial showed no or a mild PCS score (Bahmer et al. 2022, eClinicalMedicine 51:101549) at the 6-month follow-up. In order to characterize a relevant subpopulation of the risk factor population as defined above that was additionally at risk for the development of PCS, baseline characteristics and symptoms of all available patients of the placebo population (n=425) were used to identify predictors for a PCS score of ≥5 at month 6 with a significance threshold of P<0.05. These predictors were the following:
medical history: chronic obstructive pulmonary disease, organ transplantation, depression, other chronic diseases, permanent intake of medication;
symptoms from the onset of COVID-19 until the trial baseline: shortness of breath, whistling/wheezing breathing, dizziness, vomiting, conjunctivitis;
at baseline: shortness of breath, whistling/wheezing breathing, dizziness, conjunctivitis, chills, muscle pain, limb pain, limitation of activity (a score of 2 in the WHO scale of COVID-19 severity developed in 2020; https://apps.who.int/iris/rest/bitstreams/1266940/retrieve, last accessed on 20 Mar. 2023).

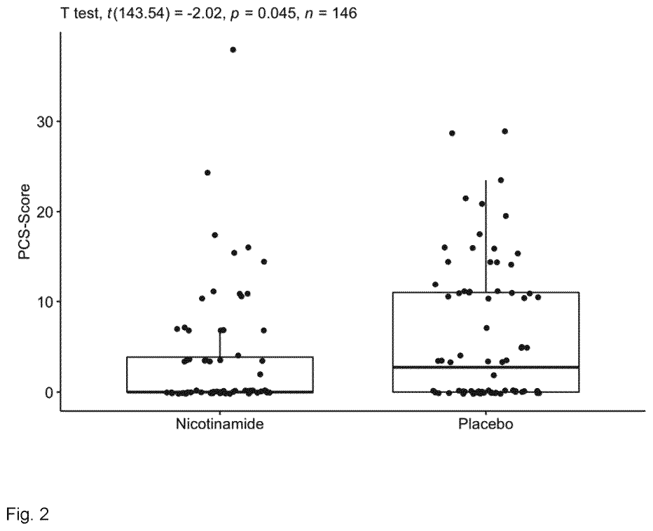

In order to be included into the PCS risk factor population, patients had to have more than 5 of these predictors at baseline. Surprisingly, nicotinamide was significantly beneficial (P=0.045; Table 7, FIG. 2) in this population with regard to the development of PCS even though only one of the symptoms (shortness of breath) had been positively affected by nicotinamide in the acute phase of the disease (56 of 92 patients treated with nicotinamide recovered at week 2 compared to 37 of 90 patients treated with placebo; P=0.012 in the Fisher Exact test).

TABLE 7
PCS score in PCS risk factor population
at 6 months after COVID-19

| | Nicotinamide | | Placebo | | | | |
|---|---|---|---|---|---|---|---|
| | N = 70 | | N = 76 | | | | |
| | | Standard | | Standard | | | |
| | Mean | Deviation | Mean | Deviation | T | df | P |
| PCS- | 3.66 | 6.72 | 6.07 | 7.73 | −2.019 | 143.543 | 0.045 |
| Score | | | | | | | |

Taken together, administration of nicotinamide surprisingly and significantly ameliorated the impaired sense of smell and/or taste in all patients of the risk factor population as well as PCS in patients that were additionally at particular risk for PCS. These effects could not be predicted from the effects of nicotinamide on acute COVID-19.

The invention claimed is:

1. A composition comprising an active substance selected from nicotinamide; nicotinic acid; nicotinic acid esters; tryptophan; a tryptophan dipeptide;
nicotinamide adenine dinucleotide (NAD); nicotinamide adenine dinucleotide phosphate (NADP); an intermediate in the biosynthesis of NAD or NADP selected from the group consisting of N-formylkynurenine, L-kynurenine, 3-hydroxy-L-kynurenine, 3-hydroxyanthranilate, 2-amino-3-carboxymuconate semialdehyde, quinolinate, nicotinic acid mononucleotide (beta-nicotinate D-ribonucleotide), and nicotinic acid adenine dinucleotide; nicotinamide riboside; nicotinamide mononucleotide; 1-methylnicotinamide (N-methylnicotinamide); or a combination thereof, for use for preventing and/or reducing one or more post-acute symptoms of an infection, wherein the composition is formulated to partly or completely release the active substance(s) for topical supplementation or efficacy in the lower small intestine and/or the colon.

2. The composition according to claim 1 for use as a pre- or post-exposure prophylaxis to prevent the onset of post-acute symptoms in patients that were at risk for an infection or tested positive for infection with the pathogen.

3. The composition according to claim 1 characterized in that the composition comprises one or more active substance formulations, preferably nicotinamide formulations, for immediate release and/or extended release and/or sustained release delivering the active substance, preferably nicotinamide, mainly systemically to the circulation together with one or more active substance formulations, preferably nicotinamide formulations, for delayed release and/or delayed-controlled release delivering the active substance, preferably nicotinamide, mainly topically to the lower small intestine and/or colon.

4. The composition according to claim 1, characterized in that the composition contains a combination of two formulation variants of active substance formulations in a specific ratio by weight in the range of from 1:1 to 1:1000.

5. The composition according to claim 1, characterized in that the composition contains a combination of two or more formulation variants of active substances in the same dosage form.

6. The composition according to claim 1, characterized in that the composition contains a variable or fixed-dose combination of two or more formulation variants of active substances in separate dosage forms.

7. The composition according to claim 1, characterized in that the composition has an active substance content, preferably nicotinamide content, of 1 to 5000 mg per finished dosage form.

8. The composition according to claim 1, wherein part of the active substance(s), preferably nicotinamide, is formulated for delayed or delayed-controlled release in order to enter the circulatory system only to a low degree, so that plasma peak levels of active substance(s) following administration of the delayed or delayed-controlled release formulation are reduced by at least 20% relative to the same amount of active substance(s) administered in immediate-release formulations in the same way and under the same conditions.

9. The composition according to claim 1, for use for administration once daily, with breakfast in the morning.

10. The composition according to claim 9, wherein the dose has an active substance content of 1 to 5000 mg per finished dosage form.

11. The composition according to claim 1, for use for preventing or reducing one or more post-acute symptoms of the infection, wherein the at least one post-acute symptom is selected from the group consisting of reduced physical performance, fatigue, exercise intolerance, chemosensory deficits, joint pain, muscle pain, chest pain, ear-nose-throat ailments, cough, wheezing, gastrointestinal ailments, neurological ailments, psychological ailments, dermatological ailments, infection signs, and sleep disturbance.

12. The composition according to claim 1, for use for administration to patients with at least one characteristic or underlying medical condition associated with an increased risk of developing severe illness from the infection and/or post-acute symptoms.

13. The composition according to claim 1, for use for administration to patients with at least one characteristic symptom of the infection.

14. The composition according to claim 1, for use for administration to patients with at least one characteristic symptom of acute COVID-19.

15. The composition according to claim 1, for use for administration in combination with foods, beverages, dietary supplements, foods for special medical purposes, probiotics, prebiotics, synbiotics and/or vitamins.
